(19) United States
(12) Patent Application Publication
(10) Pub. No.: US 2022/0084852 A1
(43) Pub. Date: Mar. 17, 2022

(54) ENVIRONMENTAL MONITORING SYSTEM

(71) Applicant: CHANGXIN MEMORY TECHNOLOGIES, INC., Hefei City (CN)

(72) Inventor: Yunxiao DING, Hefei City (CA)

(21) Appl. No.: 17/487,799

(22) Filed: Sep. 28, 2021

Related U.S. Application Data
(63) Continuation of application No. PCT/CN2021/103771, filed on Jun. 30, 2021.

(30) Foreign Application Priority Data
Sep. 16, 2020 (CN) 202010973462.7

(51) Int. Cl. H01L 21/67; G05B 23/02; H01L 21/66; G01N 33/00

(52) U.S. Cl. CPC H01L 21/67253; G01N 33/0075; H01L 22/10; G05B 23/0259

(57) ABSTRACT

The embodiments of the present application provide an environmental monitoring system, which is configured to monitor concentrations of air pollutants of process regions in a clean room, and includes: a plurality of process regions in the clean room; a sampling device, configured to collect environmental samples of the process regions; a controlling device, configured to control the sampling device to collect the preset environmental samples of the process regions; and an analyzing device, communicated with the sampling device and configured to analyze the collected environmental samples. The present application facilitates a rapid acquisition of the analysis data of the environmental samples in particular process regions and improves the accuracy of data analysis.

### CROSS-REFERENCE TO RELATED APPLICATIONS

This application is a continuation application of International Patent Application No. PCT/CN2021/103771, filed on Jun. 30, 2021, which claims priority to Chinese Patent Application No. 202010973462.7, filed with the Chinese Patent Office on Sep. 16, 2020 and entitled “ENVIRONMENTAL MONITORING SYSTEM.” International Patent Application No. PCT/CN2021/103771 and Chinese Patent Application No. 202010973462.7 are incorporated herein by reference in their entireties.

### TECHNICAL FIELD

The embodiments of the present application relate to the field of semiconductors, and in particular to an environmental monitoring system.

### BACKGROUND

With an increase in the process complexity of the semiconductor industry and the shrinking of product feature sizes, the impact of air pollutants on products has become a key concern in the aspect of environmental control for clean rooms.

The existing air pollutant detection steps are completed chiefly by workers. These detection steps can be divided into pre-processing, sampling, analysis, and data processing. It is highly likely to introduce human pollutions during the pre-processing and sampling, leading to inaccurate testing results. And during the analysis and data processing, the results of analysis obtained by different workers may be different, which causes a data accuracy issue. In addition, it takes a long time for the workers to complete the detection steps and there are issues with the timeliness of the detection results, so it is difficult to achieve rapid monitoring and processing for air pollutants.

### SUMMARY

Provided in the embodiments of the present application is an environmental monitoring system that facilitates a rapid acquisition of the analysis data of the environmental samples in particular process regions and improves the accuracy of data analysis.

To address the above problem, provided in the embodiments of the present application is an environmental monitoring system configured to monitor the concentrations of air pollutants of process regions in a clean room, which includes: a plurality of process regions in the clean room; a sampling device configured to collect environmental samples of the process regions; a controlling device configured to control the sampling device to collect the preset environmental samples of the process regions; and an analyzing device communicated with the sampling device and configured to analyze the collected environmental samples.

### BRIEF DESCRIPTION OF DRAWINGS

One or more embodiments will be exemplarily illustrated with reference to the figures in the accompanying drawings corresponding thereto. These exemplary illustrations do not constitute a limitation to the embodiments. Elements with the same reference numeral in the drawings are denoted as similar elements. Unless otherwise specifically stated, the figures in the accompanying drawings do not constitute a scale limitation.

FIG. 1 is a schematic structural diagram of the environmental monitoring system according to the embodiments of the present application;

FIG. 2 and FIG. 3 are partial schematic structural diagram of the environmental monitoring system according to the embodiments of the present application;

FIG. 4 and FIG. 5 are schematic diagrams of the operating principle of the environmental monitoring system according to the embodiments of the present application; and

FIG. 6 is a graph illustrating the test background-air pollutant concentrations according to the embodiments of the present application.

### DESCRIPTION OF EMBODIMENTS

For a better clarity of the objects, the technical solutions, and the advantages of the embodiments of the present application, a detailed description of the embodiments of the present application is given below in combination with the accompanying drawings. However, the ordinary skilled in the art can understand that many technical details are provided in the embodiments of the present application so as to make readers better understand the present application. However, even if these technical details are not provided and based on a variety of variations and modifications of the following embodiments, the technical solutions sought for protection in the present application can also be realized.

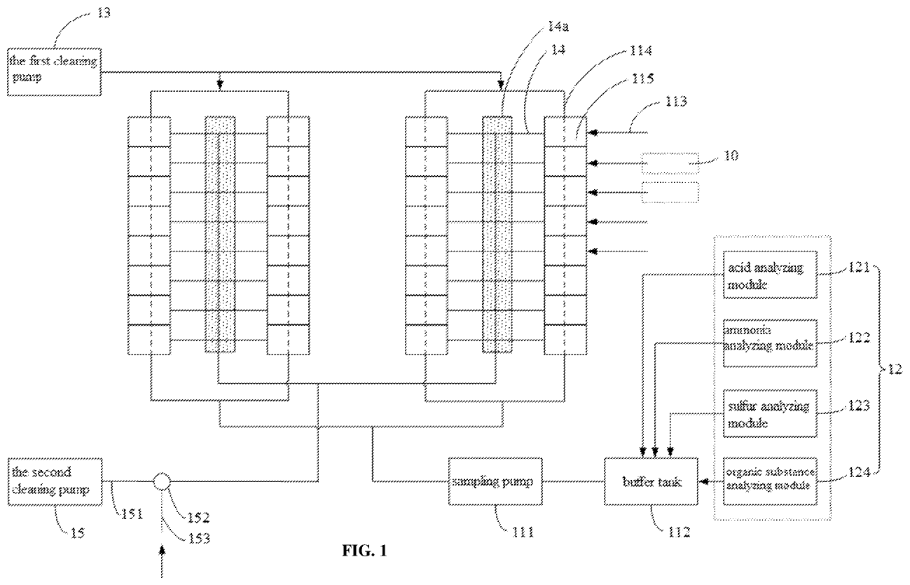

FIG. 1 is a schematic structural diagram of the environmental monitoring system according to the embodiments of the present application.

With reference to FIG. 1, the environmental monitoring system is configured to monitor the concentrations of air pollutants of process regions 10 in a clean room, and includes: a plurality of process regions 10 in the clean room; a sampling device (not illustrated) configured to collect environmental samples of the process regions 10; a controlling device (not illustrated) configured to control the sampling device to collect the preset environmental samples of the process regions 10; and an analyzing device 12 communicated with the sampling device and configured to analyze the collected environmental samples.

In this embodiment, the sampling device includes a sampling pump 111 and a buffer tank 112. The sampling pump 111 is communicated with the process regions 10, the buffer tank 112 is communicated with the analyzing device, the sampling pump 111 is configured to collect the environmental samples, and the buffer tank 112 is configured to buffer the pressure fluctuations of the environmental samples. The buffer tank 112 performs a pre-mixing stabilization on the environmental samples collected by the sampling pump 111, so it is ensured that the atmospheric pressures of the environmental samples for analysis are within a preset range, the pressure fluctuations caused by intermittent sampling of the sampling pump 111 are avoided, the data analysis of the analyzing device 12 is prevented from being influenced by the pressure fluctuations, and the accuracy of the concentration data of the air pollutants acquired by the analyzing device 12 is guaranteed.

The analyzing device 12 may carry out data analysis when the pressure inside the buffer tank 112 is within the preset range, and the buffer tank 112 may be provided with a pressure calculating function. In addition, the buffer tank 112 may have a pressure relief port that relieves a part of the pressure when the pressure inside the buffer tank 112 soars and it is too late for data analysis, thereby making ensure that the concentration data derived from the analysis has a higher accuracy.

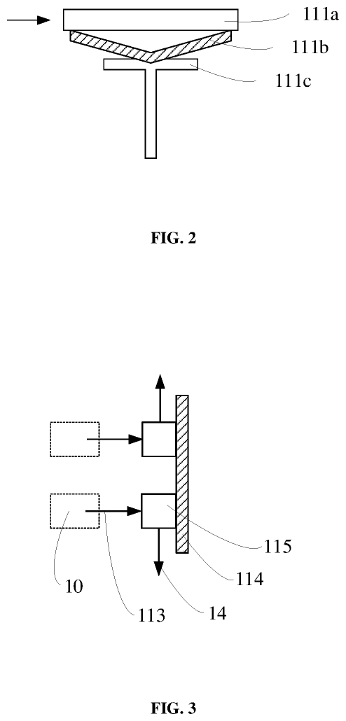

In this embodiment, the type of the sampling pump 111 includes a diaphragm pump. With reference to FIG. 2, a conveying pipeline 111a for conveying a gas is separated from a plunger 111c by a diaphragm 111b, preventing the conveyed gas from being polluted by pollutants on the surface of the plunger 111c and further guaranteeing the accuracy of the concentration data acquired by the analyzing device 12.

In this embodiment, the analyzing device 12 includes a plurality of analyzing modules, with each being configured to analyze the concentrations of the corresponding types of air pollutants. The plurality of analyzing modules can synchronously analyze the environmental samples that result from a single collection by the sampling device. As such, the concentrations of different types of air pollutants can be simultaneously acquired, with no need of successive analysis, and this helps shorten the overall analysis time for the concentrations of the air pollutants. Furthermore, the number of times of sampling for the environmental samples can be decreased to further shorten the time for monitoring the concentrations of the air pollutants, and rapid monitoring and processing of the concentrations of the air pollutants can be facilitated.

In this embodiment, the analyzing device 12 includes an acid analyzing module 121, an ammonia analyzing module 122, a sulfur analyzing module 123, and an organic substance analyzing module 124, which are respectively configured to monitor acid gas concentrations, ammonia gas concentrations, sulfur dioxide concentrations and organic substance concentrations inside the process regions 10, in order to avoid the situations where formation of metal wires is affected by the acid and ammonia gases, the product yield is affected by salts generated from the reaction of the acid and ammonia gases, the combination of sulfur dioxide and ammonia causes an atomized photomask surface, and the atomization leads to a decrease in the product yield and an increase in the rate of repetitive work.

In other embodiments, the analyzing device may also include analyzing modules for other air pollutants. It shall be noted that any gas that may affect the process or product yield can be viewed as an air pollutant, and the types of air pollutants may be different in different process regions.

In this embodiment, the controlling device may adjust the order of collections for the plurality of process regions 10 in accordance with a preset environmental requirement of at least one of the process regions 10. As such, the monitoring priorities for different process regions 10 can be adjusted to achieve important monitoring for the particular process regions.

The environmental requirement may be input manually, or imported by another apparatus. The environmental requirement may be a sampling frequency, a concentration threshold, or monitoring information. A detailed description is given below through two application examples.

The first example is as follows. In a factory, there are a plurality of clean rooms, with each having a plurality of communicable process regions 10 therein. The process regions 10 in different clean rooms may be the same, i.e., both a first clean room and a second clean room have therein a first process region configured for a first process and a second process region configured for a second process. When an air pollutant issue occurs in the first process region of the first clean room, the monitoring information of the first process region in the first clean room is imported to the controlling device of the second clean room, and the controlling device, based on the monitoring information, monitors the concentrations of the air pollutants of the first process region in the second clean room.

The second example is as follows. When a preset air pollutant concentration threshold of a process region 10 changes, the controlling device re-monitors the concentrations of the air pollutants in this process region.

In this embodiment, the controlling device is further configured to set a concentration threshold of at least one of the process regions 10. The environmental monitoring system further includes a warning device (not illustrated), which is configured to send out warning information when the concentration threshold of any process region 10 exceeds the preset concentration threshold of the process regions 10. This is favorable for a rapid cleaning of the particular process regions 10 in which the air pollutants have exceeded the standard.

In this embodiment, the sampling device includes a system sampling pipeline 114 and a plurality of single sampling pipelines 113, each process region 10 is communicated with one single sampling pipeline 113, different process regions 10 are communicated with different single sampling pipelines 113, the system sampling pipeline 114 may be communicated with any single sampling pipeline 113, and an output end of the system sampling pipeline 114 is communicated with the analyzing device 12. The sampling device further includes: sampling valves 115, which are configured to connect or disconnect the single sampling pipelines 113 with the system sampling pipeline 114.

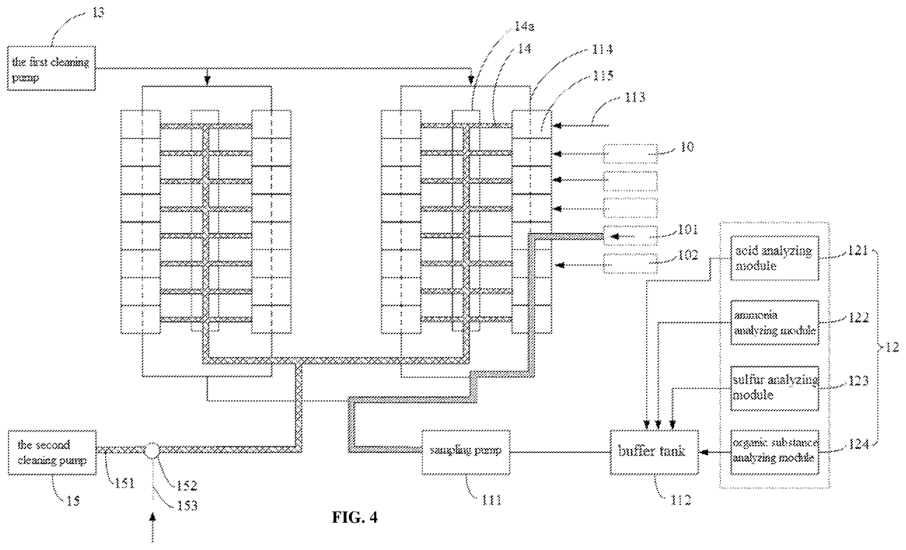

With regard to the position relationship among the single sampling pipelines 113, the system sampling pipeline 114, and the sampling valves 115, reference may be further made to FIG. 3. The system sampling pipeline 114 is a continuous channel. On an extension path of the system sampling pipeline 114, the system sampling pipeline 114 may be communicated with each single sampling pipeline 113 via the sampling valve 115. When the sampling valve 115 disconnects the single sampling pipeline 113 with the system sampling pipeline 114, the gas inside the process region 10 can only enter into the single sampling pipeline 113, rather than the system sampling pipeline 114. When the sampling valve 115 connects the single sampling pipeline 113 with the system sampling pipeline 114, the gas inside the process region 10 can reach the system sampling pipeline 114 via the single sampling pipeline 113.

It shall be noted that at the same time, there may be one or more sampling valves 115 in a state where the single sampling pipeline 113 is connected with the system sampling pipeline 114, which is to say, the analyzing device 12 can be configured not only to analyze the concentrations of the air pollutants of the single process region 10, but also to analyze the average concentration of the air pollutants of a plurality of process regions 10.

The plurality of sampling valves 115 are fixed on a valve disk 14a.

For a better understanding, the reason why the sampling pipelines are arranged as above will be described in detail below.

The reason why the plurality of single sampling pipelines 113 are arranged is that the plurality of process regions 10 need to be collected, respectively. The purpose of arranging the system sampling pipeline 114 for the communication of the plurality of sampling pipelines 113 is to reduce the number of the pipelines on a gas conveying path, and further to offer convenience for management. The environmental samples of a single process region 10 needs to be collected, so it is required that the sampling valve 115 controls the connection and disconnection between the single sampling pipeline 113 and the system sampling pipeline 114, achieving accurate collection for the particular process region 10.

Moreover, since the system sampling pipeline 114 is shared, to avoid such a situation where the next sampling is affected by the remaining air pollutants of the previous sampling procedure, the system sampling pipeline 114 needs to be cleaned between two collections. In order to avoid the situation where the air pollutants are piled up inside the single sampling pipeline 113 and thus cannot be effectively removed during the cleaning process, the sampling valve 115 may be arranged at an end of the single sampling pipeline 113 towards the system sampling pipeline 114. This helps ensure a higher cleanness in the single sampling pipeline 113 as well as a certain degree of cleaning for the sampling valve 115 itself.

In this embodiment, the environmental monitoring system includes: a first cleaning pump 13, which is configured to purge the system sampling pipeline 114 and introduce the purged gas into the analyzing device 12. As such, when the analyzing device 12 confirms that the environment of the system sampling pipeline 114 meets the sampling requirements, the controlling device may control the first cleaning pump 13 to stop purging and perform the next sampling, so as to ensure that the environment of the system sampling pipeline 114 meets the preset requirements at the time of the next sampling. As a result, interferences with the analysis of the air pollutant concentrations in the process regions 10, which are caused by the environment of the system sampling pipeline 114, are avoided, and also the accuracy of the detection results are guaranteed.

It shall be noted that the purging of the system sampling pipeline 114 by the first cleaning pump 13 also affects the adsorbability on an inner wall of the system sampling pipeline 114. In particular, the longer the purging time is, the cleaner the inner wall of the system sampling pipeline 114 becomes, so the ability of the inner wall to adsorb the air pollutants is enhanced and the detection values of the air pollutants are relatively low. Correspondingly, the shorter the purging time is, the dirtier the inner wall of the system sampling pipeline 114 becomes, so the air pollutants on the inner wall of the system sampling pipeline 114 will affect the detection results, i.e., the detection values of the air pollutants are relatively high. That is to say, while the system sampling pipeline 114 is cleaned, the cleaning time needs to be controlled such that the inner wall of the system sampling pipeline 114 has a preset level of cleanness.

In addition, a preset time threshold corresponding to the preset concentration threshold may also be set in accordance with the relationship between the air pollutant concentration values in historical data and the cleaning time of the first cleaning pump 13. In this way, the next sampling can be performed when the cleaning time is within the preset time threshold, without the need to wait for the results of analysis by the analyzing device 12. Doing so facilitates a rapid and accurate acquisition of the air pollutant concentration data in the process regions 10.

In this embodiment, the controlling device is further configured to control the valve state of the sampling valves 115, i.e., the controlling device realizes the switching of the collected targets of the process regions 10 by controlling the valve states of the sampling valves 115, thereby acquiring the air pollutant concentration data in the particular process regions 10.

In this embodiment, the environmental monitoring system further includes a cleaning pipeline 14, one end of which is configured to introduce or withdraw a gas and the other end of which may be communicated with the single sampling pipeline 113. The sampling valve 115 is configured to control the single sampling pipeline 113 to be communicated with the system sampling pipeline 114 or the cleaning pipeline 14.

In particular, with continued reference to FIG. 3, when the single sampling pipeline 113 is communicated with the cleaning pipeline 14, the sampling valve 115 disconnects the single sampling pipeline 113 with the system sampling pipeline 114, and the gas inside the system sampling pipeline 114 cannot be introduced into the single sampling pipeline 113 or the corresponding process region 10, in which case the single sampling pipeline 113 is cleaned through the cleaning pipeline 14. As such, it can be ensured that the single sampling pipeline 113 has a higher cleanness and accordingly interferences with the analysis results of the air pollutant concentrations in the process regions 10, which are caused by the air pollutants inside the single sampling pipeline 113, are avoided.

In this embodiment, the environmental monitoring system further includes: a gas exchange valve 152, an intake pipeline 153, an exhaust pipeline 151, and a second cleaning pump 15. The gas exchange valve 152 is configured to control one end of the cleaning pipeline 14 to be communicated with one end of the intake pipeline 153, and the other end of the intake pipeline 153 is configured to introduce a clean gas; alternatively, the gas exchange valve 152 is configured to control one end of the cleaning pipeline 14 to be communicated with one end of the exhaust pipeline 151, the other end of the exhaust pipeline 151 is communicated with the second cleaning pump 15, and the second cleaning pump 15 is configured for gas withdrawal.

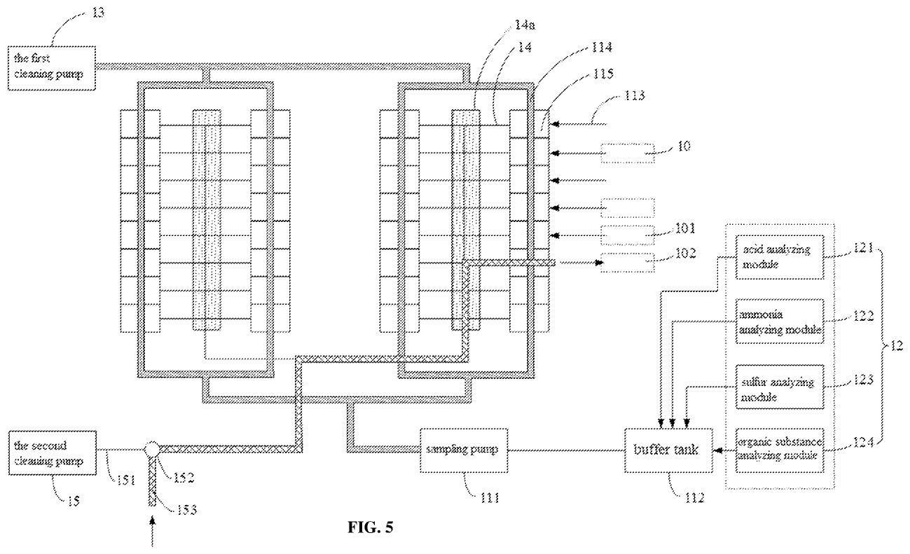

The cleaning effects brought by gas withdrawal and gas introduction as well as the timings of application of gas withdrawal and gas introduction are different. By referring to FIG. 4 and FIG. 5, with the sequential samplings for the first process region 101 and the second process region 102 as an example, a detailed description is given below.

With reference to FIG. 4, when the sampling is performed for the first process region 101, the single sampling pipelines 113 corresponding to other process regions 10 may be communicated to the cleaning pipeline 14, one end of the cleaning pipeline 14 is communicated to the exhaust pipeline 151, and the second cleaning pump 15, by means of gas withdrawal, causes the gases inside the single sampling pipelines 113 to be identical to the environmental gases inside the corresponding process regions 10, which thus facilitates the next sampling. Also, the gases inside the single sampling pipelines 113 are kept in a flowing state to avoid the situation where the air pollutants inside the single sampling pipelines 113 are adsorbed onto the inner walls of the single sampling pipelines 113 because the gases are stationary, so a higher cleanness inside the single sampling pipelines 113 is ensured and the accuracy of the detection results can be advantageously improved.

It is to be understood that during the course of the sampling for the first process region 101, the surface of each sampling valve, which is exposed by the system sampling pipeline 114, has the air pollutants deposited thereon due to the contact of the system sampling pipeline 114 with each sampling valve. Meanwhile, a small amount of air pollutants will be transferred to other locations of the sampling valves 115 since the air pollutants have small molecules and are susceptible to drifting. However, when the first cleaning pump 13 is subsequently employed to clean the system sampling pipeline 114, only the air pollutants on the surface of the sampling valve 115 exposed by the system sampling pipeline 114 can be removed. That is, the small amount of air pollutants transferred to the other locations cannot be removed.

With reference to FIG. 5, Upon completion of the sampling for the first process region 101 and prior to the sampling for the second process region 102, it is required that the first cleaning pump 13 is utilized to purge the system sampling pipeline 114 to remove the air pollutants that remain inside the system sampling pipeline 114 at the time of the sampling for the first process region 101, and doing so avoids the influence of the air pollutants inside the system sampling pipeline 114 on the results of analysis by the analyzing device 12. In addition, since a small amount of air pollutants cannot be purged by the first cleaning pump 13, opposite purging may be performed by introducing clean dry air (CDA), such that the single sampling pipeline 113 corresponding to the second process region 102 is further cleaned. This opposite purging has a little influence on the results of analysis owing to the small amount of the remaining air pollutants.

With respect to the opposite purging-type cleaning, the gas withdrawal-type cleaning may withdraw different air pollutants inside the process regions 10 in an equal or unequal proportion manner, leading to inaccurate detection results. In particular, when the proportion of the withdrawn air pollutants in all the air pollutants is different from the proportion of the withdrawn carrier gases (other gases than the air pollutants) in all the carrier gases, the air pollutant concentrations inside the process regions 10 will increase or decrease; and when the proportions of different types of withdrawn air pollutants in the total quantity of these types of air pollutants are different, the relative relationships of the concentrations of different types of air pollutants inside the process regions 10 will change.

The total quantity of the clean dry gas introduced at the time of opposite purging is calculatable, so with the opposite purging-type cleaning scheme employed, the influence of the clean dry gas on the air pollutant concentration analysis can be eliminated through calculations and thus the results of analysis of the air pollutants can be accurately obtained. The results of analysis include the types of air pollutants, the concentrations of air pollutants, and the concentration ratios of different air pollutants.

It shall be noted that since the gas utilized by the first cleaning pump 13 for purging is introduced into the buffer tank in the end, an inert gas may be adopted for purging. And since the gas at the time of opposite purging is introduced into the process regions 10 of the clean room in the end and there may be workers within this clean room, it is preferable to employ the breathable dry air to ensure the safety of the workers. Furthermore, the gas for opposite purging may be introduced into other regions by regulating the structures of the single sampling pipelines 113 or by regulating the position relationships of the single sampling pipelines 113 and the sampling valve 115, thus further ensuring the accuracy of the concentration data obtained by the analyzing device.

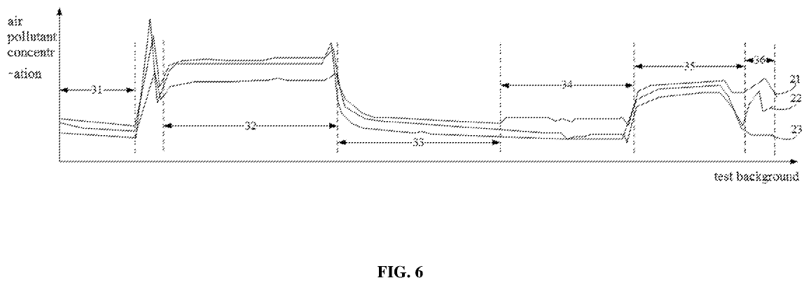

For a better understanding, the detection results under different test backgrounds will be described below in details with reference to FIG. 6.

Referring to FIG. 6, the concentrations of the air pollutants in three process regions 10 (see FIG. 1), i.e., a large yellow light region 21, a small yellow light region 22 and a photomask room 23, are tested.

In a first test background 31, the concentrations of the air pollutants in the three process regions 10 are directly tested. At this moment, due to the relatively low concentrations of the air pollutants in various process regions 10 and the higher cleanness on the inner walls of the system sampling pipeline 114 and of the sampling valves 115, the concentration detection results indicate that the concentrations of the air pollutants in all the three process regions 10 are relatively low and there is no significant distinctiveness.

In a second test background 32, as a large amount of air pollutants remain inside the system sampling pipeline 114 and the sampling valves 115, the detection results of the concentrations of the air pollutants in each process region 10 is affected, demonstrating that the concentrations of the air pollutants are relatively high.

In a third test background 33, the remaining air pollutants are partially removed by opposite purging of the sampling valves 115 with the clean dry air as well as by purging of the system sampling pipeline 114 with a nitrogen gas, such that the detection results show that the concentrations of the air pollutants in each process region 10 are relatively low.

In a fourth test background 34, the excessively long time for purging with the nitrogen gas, which is approximately 1200 s, results in a stronger adsorbability on the inner wall of the system sampling pipeline 114, so the detection results show that the concentrations of the air pollutants in each process region 10 are relatively low, and there is no distinctiveness.

In a fifth test background 35, due to the very short purging time that is about 300 s, the influence from the remaining pollutants is still large, the detection results show that the concentrations in the three process regions 10 are relatively high, and the pollutions in the three process regions 10 cannot be distinguished in an effective way.

In a sixth test background 36, the purging time is controlled to be 600 s, or to be ¼ to ⅙ of the sampling time, such that the detection results of the concentrations of the air pollutants in different process regions 10 differ vastly, and there is significant distinctiveness. The process regions 10 included in the sixth test background 36 has relatively low concentrations of the air pollutants.

In this embodiment, manual sampling and analysis are replaced by the sampling device and the analyzing device, which helps acquire the analysis data of the environmental samples rapidly and carry out corresponding processing. Also, this contributes to avoiding the introduction of human pollutions and makes sure that the analysis data has a higher accuracy. The fact that the sampling device is controlled by the controlling device to collect the environmental samples facilitates an accurate acquisition of the analysis data of a particular process region or the average analysis data of a plurality of particular process regions, i.e., the analysis data of the environmental samples in the particular process regions can be acquired in a rapid way.

Ordinary skilled in the art can understand that the implementations described above are particular embodiments for implementing the present application. In practical uses, various changes in forms and details may be made to the implementations without departing from the spirit and scope of the present application. Any skills in the art may make their own changes and modifications without departing from the spirit and scope of the present application. Therefore, the protection scope of the present application shall be subject to the protection scope of the claims.

What is claimed is:

1. An environmental monitoring system, configured to monitor concentrations of air pollutants of process regions in a clean room, wherein the environmental monitoring system comprises: a plurality of process regions in the clean room;
a sampling device, configured to collect environmental samples of the process regions;
a controlling device, configured to control the sampling device to collect the environmental samples of the process regions; and
an analyzing device, communicated with the sampling device and configured to analyze the collected environmental samples.

2. The environmental monitoring system according to claim 1, wherein the sampling device comprises a sampling pump and a buffer tank, the sampling pump is communicated with the process regions, the buffer tank is communicated with the analyzing device, the sampling pump is configured to collect the environmental samples, and the buffer tank is configured to buffer pressure fluctuations of the environmental samples.

3. The environmental monitoring system according to claim 2, wherein a type of the sampling pump comprises a diaphragm pump.

4. The environmental monitoring system according to claim 1, wherein the analyzing device comprises a plurality of analyzing modules, each of the analyzing modules is configured to analyze the concentrations of the corresponding types of air pollutants, and the plurality of analyzing modules synchronously analyze the environmental samples that result from a single collection by the sampling device.

5. The environmental monitoring system according to claim 1, wherein the analyzing device comprises an acid analyzing module, an ammonia analyzing module, a sulfur analyzing module, and an organic substance analyzing module.

6. The environmental monitoring system according to claim 1, wherein the controlling device adjusts an order of collections for the plurality of process regions in accordance with a preset environmental requirement of at least one of the process regions.

7. The environmental monitoring system according to claim 1, wherein the controlling device is further configured to set a concentration threshold of at least one of the process regions; the environmental monitoring system further comprises a warning device, which is configured to send out warning information when the concentration threshold of any of the process regions exceeds the concentration threshold of the process regions.

8. The environmental monitoring system according to claim 1, wherein the sampling device comprises a system sampling pipeline and a plurality of single sampling pipelines, each of the process regions is communicated with one of the single sampling pipelines, different process regions are communicated with different single sampling pipelines, the system sampling pipeline is communicated with any of the single sampling pipelines, and an output end of the system sampling pipeline is communicated with the analyzing device; the sampling device further comprises: sampling valves, which are configured to connect or disconnect the single sampling pipelines with the system sampling pipeline.

9. The environmental monitoring system according to claim 8, wherein the environmental monitoring system further comprises: a first cleaning pump, which is configured to purge the system sampling pipeline and introduce purged gas into the analyzing device.

10. The environmental monitoring system according to claim 8, wherein the controlling device is further configured to control a valve state of the sampling valve.

11. The environmental monitoring system according to claim 8, wherein the environmental monitoring system further comprises a cleaning pipeline, a first end of the cleaning pipeline is configured to introduce or withdraw a gas, a second end of the cleaning pipeline is communicated with the single sampling pipeline, and the sampling valve is configured to control the single sampling pipeline to be communicated with the system sampling pipeline or the cleaning pipeline.

12. The environmental monitoring system according to claim 11, wherein the environmental monitoring system further comprises: a gas exchange valve, an intake pipeline, an exhaust pipeline and a second cleaning pump, the gas exchange valve is configured to control one end of the cleaning pipeline to be communicated with one end of the intake pipeline, and the other end of the intake pipeline is configured to introduce a clean gas; or, the gas exchange valve is configured to control one end of the cleaning pipeline to be communicated with one end of the exhaust pipeline, the other end of the exhaust pipeline is communicated with the second cleaning pump, and the second cleaning pump is configured for gas withdrawal.

13. The environmental monitoring system according to claim 4, wherein the analyzing device comprises an acid analyzing module, an ammonia analyzing module, a sulfur analyzing module, and an organic substance analyzing module.
